(19) United States
(12) Patent Application Publication
(10) Pub. No.: US 2016/0090373 A1
(43) Pub. Date: Mar. 31, 2016

(54) SOLID FORMS OF ANTI-VIRAL COMPOUNDS

(71) Applicant: AbbVie Inc., North Chicago, IL (US)

(72) Inventors: Jean-Christophe Califano, Whitefish Bay, WI (US); Michael G. Fickes, Lake Bluff, IL (US); Lars F. Nordstroem, Evanston, IL (US)

(21) Appl. No.: 14/867,785

(22) Filed: Sep. 28, 2015

Related U.S. Application Data
(60) Provisional application No. 62/056,746, filed on Sep. 29, 2014.

(51) Int. Cl. C07D 401/14

(52) U.S. Cl. CPC C07D 401/14

(57) ABSTRACT

Disclosed are crystalline forms of a compound of formula (I), methods of their preparation, and related pharmaceutical preparations thereof.

### RELATED APPLICATIONS

This application claims the benefit of priority to U.S. Provisional Patent Application Ser. No. 62/056,746, filed Sep. 29, 2014.

### BACKGROUND

Hepatitis C Virus (HCV) is an RNA virus belonging to the Hepacivirus genus in the Flaviviridae family. The enveloped HCV virion contains a positive stranded RNA genome encoding all known virus-specific proteins in a single, uninterrupted, open reading frame. The open reading frame comprises approximately 9500 nucleotides and encodes a single large polyprotein of about 3000 amino acids. The polyprotein comprises a core protein, envelope proteins E1 and E2, a membrane bound protein p7, and the non-structural proteins NS2, NS3, NS4A, NS4B, NS5A and NS5B.

The nonstructural protein NS5A is a membrane-associated phosphoprotein present in basally phosphorylated and hyperphosphorylated forms. It is a critical component of HCV replication and is believed to exert multiple functions at various stages of the viral life cycle. A full-length NS5A protein comprises three domains—namely, Domain I, Domain II, and Domain III. Domain I (residues 1 to 213) contains a zinc-binding motif and an amphipathic N-terminal helix which can promote membrane association. Domain II (residues 250 to 342) has regulatory functions, such as interactions with protein kinase PKR and PI3K, as well as NS5B, and also contains the interferon sensitivity-determining region. Domain III (residues 356 to 447) plays a role in infectious virion assembly, and can be modulated by phosphorylation within the domain. NS5A has been identified as a promising therapeutic target for treating HCV.

Compounds of formula (I) (PCT Application Publication No. WO 2012/051361; incorporated by reference) inhibit the replication of HCV and are therefore useful for treating HCV infection.

### SUMMARY OF INVENTION

One aspect of the invention relates to a crystalline compound having the structure of formula (I),

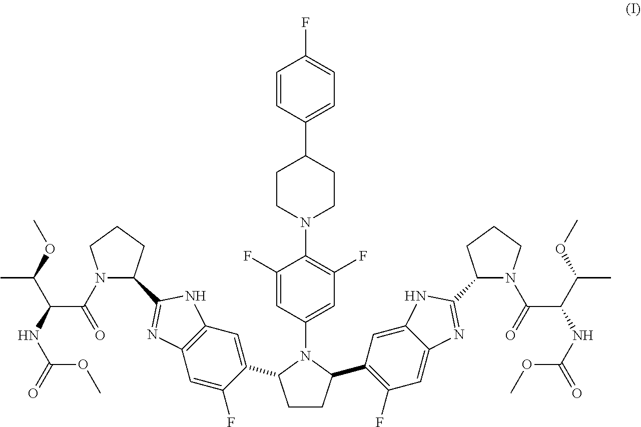 [SMILES: COC(=O)N[C@H](C(=O)N1CCC[C@H]1c1nc2cc(F)c([C@H]3CC[C@H](c4cc5[nH]c([C@@H]6CCCN6C(=O)[C@@H](NC(=O)OC)[C@@H](C)OC)nc5cc4F)N3c3cc(F)c(N4CCC(c5ccc(F)cc5)CC4)c(F)c3)cc2[nH]1)[C@@H](C)OC]

or a crystalline solvate thereof.

Another aspect of the invention relates to methods for preparing the crystalline compounds and crystalline solvates of the compound of formula (I).

In certain embodiments, the present invention provides a pharmaceutical preparation suitable for use in a human patient, comprising a crystalline compound of of formula (I) and one or more pharmaceutically acceptable excipients.

### BRIEF DESCRIPTION OF THE DRAWINGS

FIG. 1 is a thermogravimetric analysis (TGA) thermogram of the n-butylamine-H₂O solvate of formula (I) (Pattern A) at 10° C./min.

FIG. 2A is a powder X-ray diffraction (XRD) pattern of the n-butylamine-H₂O solvate of formula (I) (Pattern A). The calculated XRD pattern is shown on top, and the experimental XRD pattern is shown on bottom.

FIG. 2B is a tabulation of the peak values and relative intensities from the experimental XRD pattern shown in FIG. 2A.

FIG. 3 is a TGA thermogram of the methyl ethyl ketone-heptane solvate of formula (I) (Pattern B) at 10° C./min.

FIG. 4A is a powder XRD pattern of the methyl ethyl ketone-heptane solvate of formula (I) (Pattern B).

FIG. 4B is a tabulation of the peak values and relative intensities from the experimental XRD pattern shown in FIG. 4A.

FIG. 5 is a TGA thermogram of the methanol-diethyl ether solvate of formula (I) (Pattern B) at 10° C./min.

FIG. 6A is a powder XRD pattern of the methanol-diethylether solvate of formula (I) (Pattern B).

FIG. 6B is a tabulation of the peak values and relative intensities from the XRD pattern shown in FIG. 6A.

FIG. 7 is a TGA thermogram of formula (I) anhydrate (Pattern C) at 10° C./min.

FIG. 8A is a powder XRD pattern of formula (I) anhydrate (Pattern C).

FIG. 8B is a tabulation of the peak values and relative intensities from the XRD pattern shown in FIG. 8A.

FIG. 9 is a TGA thermogram of formula (I) MTBE solvate (Pattern C) at 10° C./min.

FIG. 10A is a powder XRD pattern of formula (I) MTBE solvate (Pattern C).

FIG. 10B is a tabulation of the peak values and relative intensities from the XRD pattern shown in FIG. 10A.

FIG. 11A is a powder XRD pattern of formula (I) EtOH—H₂O solvate (Pattern D).

FIG. 11B is a tabulation of the peak values and relative intensities from the XRD pattern shown in FIG. 11A.

FIG. 12 is a TGA thermogram of formula (I) hydrate (Pattern E) at 10° C./min.

FIG. 13A is a powder XRD pattern of formula (I) hydrate (Pattern E).

FIG. 13B is a tabulation of the peak values and relative intensities from the XRD pattern shown in FIG. 13A.

FIG. 14A is a powder XRD pattern of formula (I) acetonitrile solvate (Pattern F).

FIG. 14B is a tabulation of the peak values and relative intensities from the XRD pattern shown in FIG. 14A.

FIG. 15 is a TGA thermogram of formula (I) anhydrate (Pattern G) at 10° C./min.

FIG. 16A is a powder XRD pattern of formula (I) anhydrate (Pattern G).

FIG. 16B is a tabulation of the peak values and relative intensities from the XRD pattern shown in FIG. 16A.

FIG. 17 is a TGA thermogram of formula (I) di-n-butyl ether solvate (Pattern H) at 10° C./min.

FIG. 18A is a powder XRD pattern of formula (I) di-n-butyl ether solvate (Pattern H).

FIG. 18B is a tabulation of the peak values and relative intensities from the XRD pattern shown in FIG. 18A.

### DETAILED DESCRIPTION OF THE INVENTION

### I. Description of the Invention

The present invention is based on the surprising discovery that a compound of formula (I) can be crystallized in a solvent system comprising n-butylamine Such crystals can be used to seed saturated or supersaturated solutions of the compound of formula (I) in other solvents and other solvent systems to obtain further crystalline forms and crystalline solvates, as described herein.

Accordingly, in certain embodiments, the invention provides a crystalline compound having the structure of formula (I),

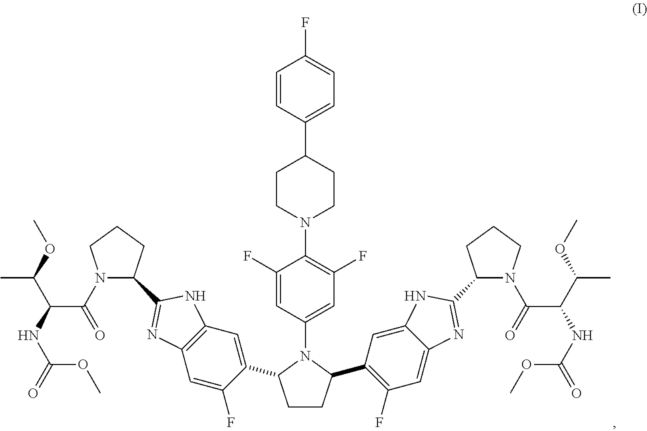 [SMILES: COC(=O)N[C@H](C(=O)N1CCC[C@H]1c1nc2cc(F)c([C@H]3CC[C@H](c4cc5[nH]c([C@@H]6CCCN6C(=O)[C@@H](NC(=O)OC)[C@@H](C)OC)nc5cc4F)N3c3cc(F)c(N4CCC(c5ccc(F)cc5)CC4)c(F)c3)cc2[nH]1)[C@@H](C)OC]

or a crystalline solvate thereof

In certain embodiments, the present invention provides a pharmaceutical preparation comprising a crystalline compound of formula (I) and one or more pharmaceutically acceptable excipients. In certain embodiments, the pharmaceutical preparations may be used for treating or preventing a condition or disease as described herein.

In certain embodiments, the present invention relates to methods of treating hepatitis C virus comprising administering a crystalline form of a compound of formula (I) to a patient.

In certain embodiments, the present invention relates to methods of making a crystalline form of a compound of formula (I) or a crystalline solvate thereof. The methods of making a crystalline form of a compound of formula (I) comprise contacting a compound of formula (I) with one or more recrystallization solvents and crystallizing the compound of formula (I), e.g., by contacting the solution with a crystal of the compound of formula (I).

Any crystalline compound thereof described herein may be used in the manufacture of a medicament for the treatment of any diseases or conditions disclosed herein.

### II. Crystal Forms of the Invention

In certain embodiments, the invention is a crystalline compound having the structure of formula (I),

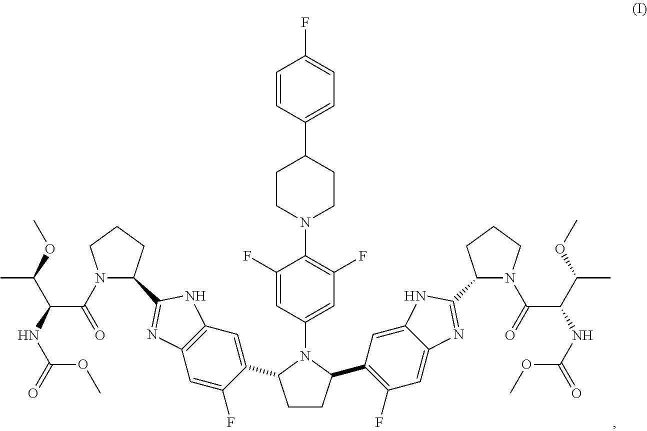 [SMILES: COC(=O)N[C@H](C(=O)N1CCC[C@H]1c1nc2cc(F)c([C@H]3CC[C@H](c4cc5[nH]c([C@@H]6CCCN6C(=O)[C@@H](NC(=O)OC)[C@@H](C)OC)nc5cc4F)N3c3cc(F)c(N4CCC(c5ccc(F)cc5)CC4)c(F)c3)cc2[nH]1)[C@@H](C)OC]

or crystalline solvates thereof

Methods of preparation and characterization data for the compound of formula (I) are found in PCT Application Publication No. WO 2012/051361, the contents of which are incorporated herein by reference.

In certain embodiments, the crystalline compound of formula (I) is not associated with a solvent molecule in its crystal lattice. In certain such embodiments, where the crystalline compound does not include water or solvent molecules in the crystal lattice, the crystalline compound may be referred to herein as an “anhydrate”.

In certain embodiments, the crystalline compound is a crystalline solvate of a compound of formula (I). In certain embodiments, the crystalline solvate includes molecules of one type of solvent compound in the crystalline solvate complex. In certain embodiments, the crystalline solvate includes molecules of one, or more than one type of solvent compound in the crystalline solvate complex. Exemplary solvent compounds that may be present in the crystalline solvate complexes of the invention include, but are not limited to, n-butylamine, propylamine, amylamine, n-hexylamine, sec-butylamine, isobutylamine, acetonitrile, methyl ethyl ketone (MEK), methanol, ethanol, n-butanol, 2-butanol, n-pentanol, diethyl ether, di-n-butyl ether, methyl ten-butyl ether, heptane, pentane, cyclohexane, acetone, ethyl acetate, isopropanol, water, or any combination thereof. In preferred embodiments, solvent compounds that are part of the crystalline solvate complexes of the invention include, but are not limited to, n-butylamine, acetonitrile, methyl ethyl ketone (MEK), methanol, diethylether, methyl tert-butylether, heptane, water, or any combination thereof. A crystalline solvate including water molecules in the crystal lattice is alternatively referred to herein as a “hydrate”.

In certain embodiments, the crystalline compounds and solvates thereof of the present invention can assemble into more than one crystal lattice. In certain such embodiments, the different crystal lattices may be referred to herein as “Pattern A”, “Pattern B” and the like.

In certain embodiments, the crystal form of the compound or crystalline solvate is characterized by powder X-ray diffraction (XRD). θ represents the diffraction angle, measured in degrees. In certain embodiments, the diffractometer used in XRD measures the diffraction angle as two times the diffraction angle θ. Thus, in certain embodiments, the diffraction patterns described herein refer to X-ray intensity measured against angle 2θ.

In certain preferred embodiments, the crystalline compound of formula (I) is an anhydrate. In certain embodiments, the crystalline anhydrate of formula (I) is referred to herein as Pattern G, and has 2θ values 5.31; 12.60; 13.75; 17.62; and 21.30. In further embodiments, Pattern G has 2θ values 5.31; 11.11; 12.60; 13.75; 15.96; 17.62; 19.71; 21.30; and 22.88. In yet further embodiments, Pattern G has 2θ values 5.31; 10.16; 10.62; 11.11; 12.60; 13.75; 15.29; 15.96; 17.62; 18.19; 19.71; 21.30; 22.88 and 26.40. In yet further embodiments, Pattern G has 2θ values 5.31; 10.16; 10.62; 11.11; 12.60; 13.75; 15.29; 15.96; 17.62; 18.19; 19.16; 19.71; 20.58; 21.30; 22.40; 22.88; 23.66; 26.40; 26.74; 28.12; 31.62; and 33.46.

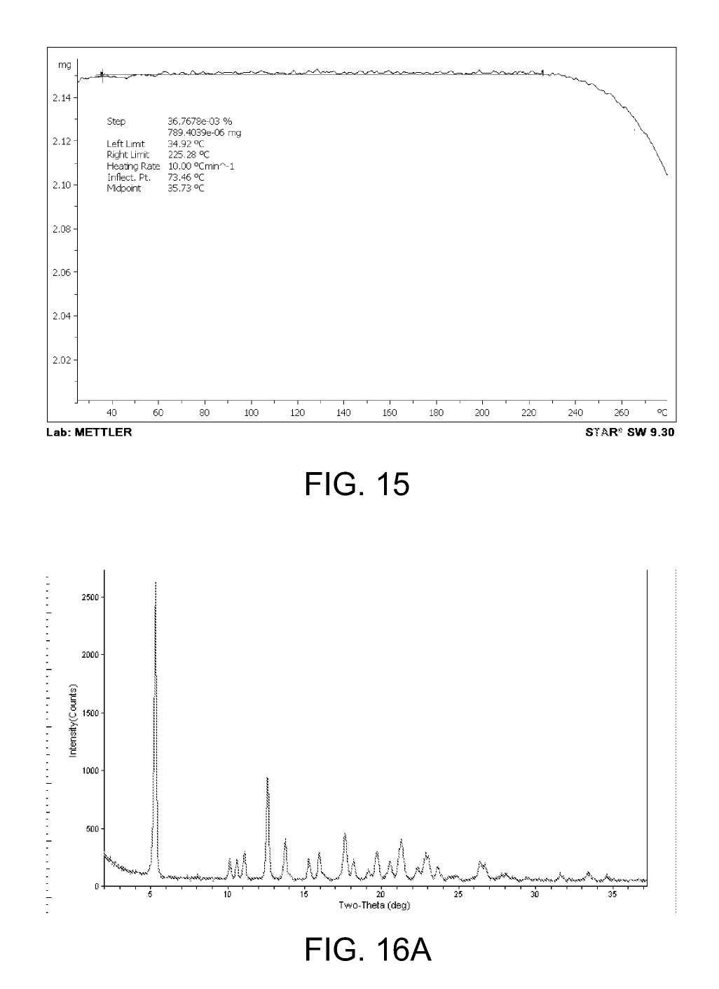

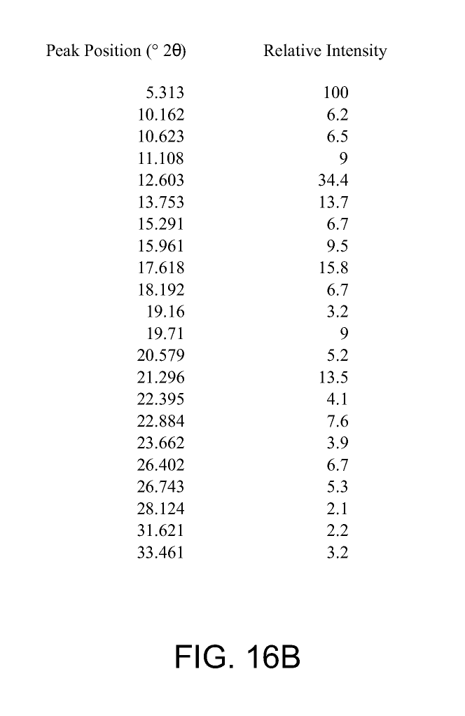

In certain embodiments, Pattern G of the crystalline compound of formula (I) has an XRD pattern substantially as shown in FIG. 16A.

In certain embodiments, the crystal form of the compound or crystalline solvate is characterized by thermogravimetric analysis (TGA), which measures a physical or chemical change of the crystalline form as a function of temperature. In certain embodiments, TGA measures the stability of a crystal form as a function of temperature. In certain such embodiments, the stability of a crystal form is gauged by mass loss of the crystal form. A plot of this data is referred to as a “thermogram”.

In certain embodiments, Pattern G of the crystalline compound of formula (I) has a TGA thermogram substantially as shown in FIG. 15.

In certain preferred embodiments, the crystalline compound of formula (I) is an anhydrate. In certain embodiments, the crystalline compound of formula (I) is a hydrate. In certain embodiments, the crystalline anhydrate of formula (I) is referred to herein as Pattern C, and has 2θ values 5.43; 6.24; 7.53; 13.67; and 19.30. In further embodiments, Pattern C has 2θ values 5.43; 6.24; 7.53; 10.91; 12.34; 12.57; 13.67; 13.94; 17.44; and 19.30. In yet further embodiments, Pattern C has 2θ values 5.43; 6.24; 7.53; 8.68; 10.91; 12.34; 12.57; 13.67; 13.94; 16.64; 17.44; 19.30; 21.10; 21.33; and 21.72. In yet further embodiments, Pattern C has 2θ values 5.43; 6.24; 7.53; 8.68; 9.28; 10.58; 10.91; 11.65; 12.34; 12.57; 13.67; 13.94; 14.71; 15.40; 15.99; 16.64; 17.44; 17.62; 19.30; 19.70; 20.90; 21.10; 21.33; 21.72; and 22.78.

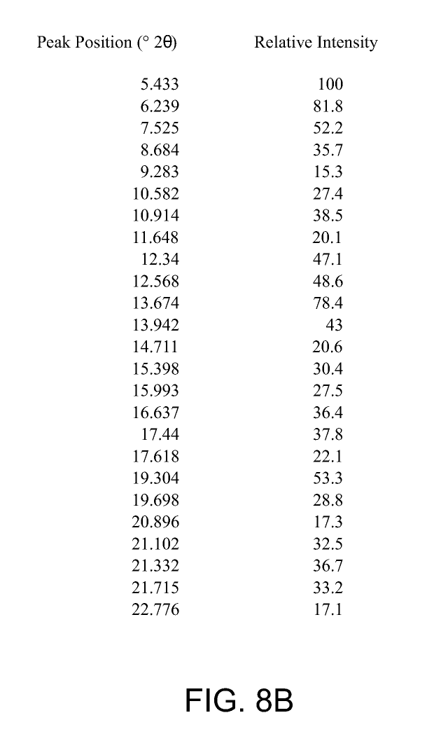

In certain embodiments, Pattern C of the crystalline compound has an XRD pattern substantially as shown in FIG. 8A.

In certain embodiments, a crystalline compound of formula (I) is not solvated (e.g., the crystal lattice does not comprise molecules of a solvent). In certain alternative embodiments, a crystalline compound of formula (I) is solvated.

In certain embodiments, the invention relates to crystalline solvates of the compound of formula (I). In certain such embodiments, the crystalline solvates comprise one or more solvent compounds selected from n-butylamine, propylamine, amylamine, n-hexylamine, sec-butylamine, isobutylamine, acetonitrile, methyl ethyl ketone (MEK), methanol, ethanol, n-butanol, 2-butanol, n-pentanol, diethyl ether, di-n-butyl ether, methyl tert-butyl ether, heptane, pentane, cyclohexane, acetone, ethyl acetate, isopropanol, and water.

The asymmetric unit of the crystalline solvate complex, as solved by X-ray diffraction, can comprise one or more solvent molecules. In certain embodiments, the crystalline solvate complex comprises one solvent compound, for example, acetonitrile, methyl tertbutyl ether, water, or di-n-butyl ether. In other embodiments, the crystalline solvate complex comprises one or more different solvent compounds. In certain embodiments, the crystalline solvate complex comprises one solvent compound, or two or three different solvent compounds, for example methanol and diethyl ether, or methyl ethyl ketone and heptane.

In certain embodiments of the invention, the crystalline solvate is an acetonitrile solvate. The acetonitrile solvate is sometimes referred to herein as Pattern F. In certain embodiments, the acetonitrile solvate of the invention has 2θ values 5.08; 12.05; 21.73; and 25.53. In certain embodiments, the acetonitrile solvate of the invention has 2θ values 5.08; 10.81; 12.05; 13.47; 19.48; 21.73; and 25.53. In further embodiments, the acetonitrile solvate Pattern F has 2θ values 5.08; 10.81; 12.05; 13.47; 13.68; 17.68; 19.02; 19.48; 20.36; 21.73; 22.24; 23.48; and 25.53. In yet further embodiments, the acetonitrile solvate has 2θ values 5.08; 7.82; 10.27; 10.81; 11.11; 12.05; 13.47; 13.68; 14.95; 15.57; 16.28; 16.81; 17.68; 19.02; 19.48; 20.36; 21.73; 22.24; 23.48; 24.16; 25.53; 26.93; 28.26; 30.41; 31.07; 32.01; 33.12; and 35.04.

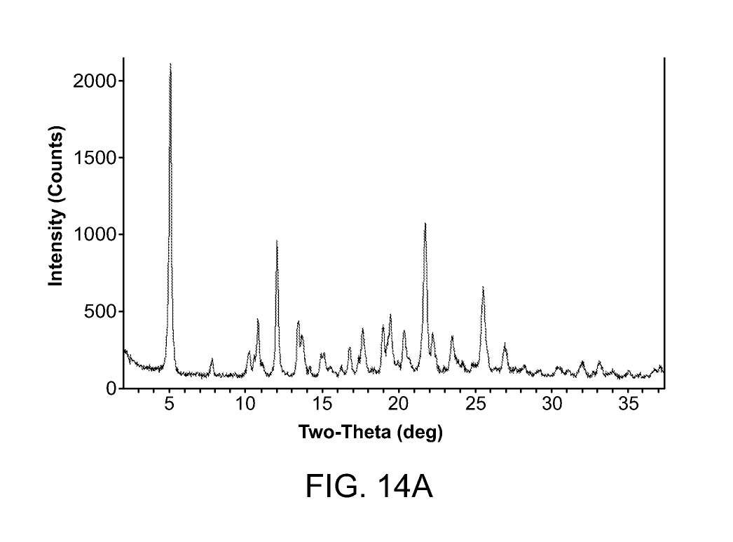

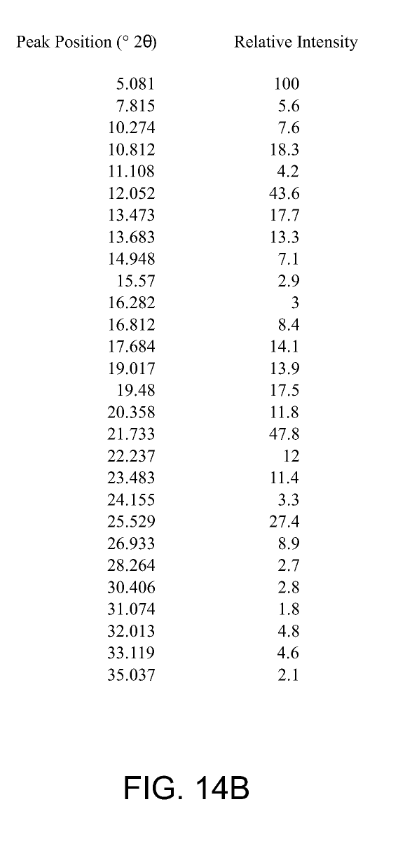

In certain embodiments, the acetonitrile solvate Pattern F of the crystalline compound has an XRD pattern substantially as shown in FIG. 14A.

In certain embodiments, the crystalline solvate is a methanol and diethyl ether solvate. The methanol-diethyl ether solvate is sometimes referred to herein as Pattern B. In certain embodiments, the methanol-diethyl ether solvate has 2θ values 5.69; 10.49; 11.38; 17.23; and 21.41. In certain embodiments, the methanol-diethyl ether solvate has 2θ values 5.22; 5.69; 7.55; 8.21; 10.49; 11.38; 11.84; 15.99; 17.23; 19.18; and 21.41. In further embodiments, the methanol-diethyl ether solvate has 2θ values 5.22; 5.69; 7.55; 8.21; 10.49; 11.38; 11.84; 12.04; 12.67; 13.24; 15.99; 17.23; 19.18; 20.15; 21.41; 22.10; 23.02; and 25.19. In yet further embodiments, the methanol-diethyl ether solvate has 2θ values 5.22; 5.69; 7.55; 8.21; 8.99; 9.40; 10.49; 11.07; 11.38; 11.84; 12.04; 12.67; 13.24; 13.99; 14.96; 15.99; 17.23; 18.10; 18.47; 19.18; 20.15; 21.41; 22.10; 22.53; 23.02; 25.19; 25.69; 26.57; 26.98; 30.09; and 32.45.

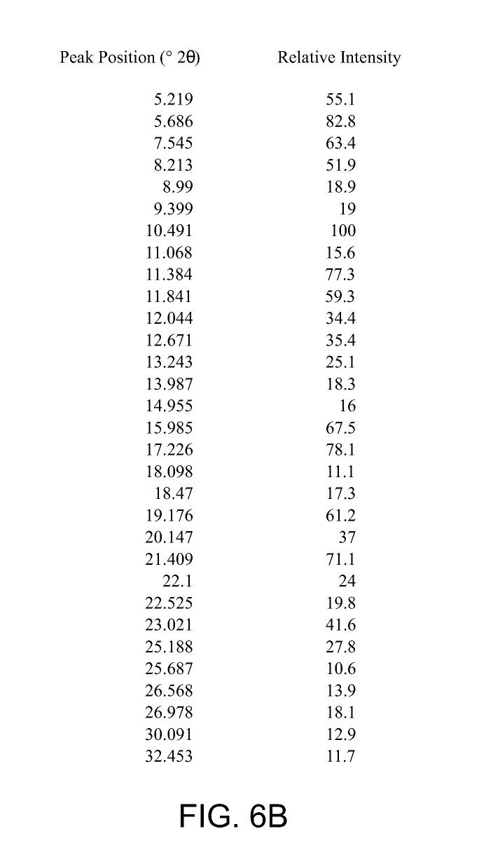

In certain embodiments, the methanol-diethyl ether solvate Pattern B of the crystalline compound has an XRD pattern substantially as shown in FIG. 6A.

Further anhydrate and solvate crystalline forms of the compound of formula (I) are described in the Examples.

In certain embodiments, the crystalline compound of formula (I) may include prodrugs of the compound of formula (I), e.g., wherein a C(O)—NH moiety in the parent compound is derivatized to replace the hydrogen atom of the amide with a group that can be hydrolyzed or otherwise cleaved to restore the C(O)—NH moiety. In certain such embodiments, the prodrug is metabolized to the active parent compound in vivo.

In certain embodiments, the crystalline compound of formula (I) may include salts of the compound of formula (I). Depending on the particular compound, a salt of a compound may be advantageous due to one or more of the salt's physical properties, such as enhanced pharmaceutical stability under certain conditions or desired solubility in water or oil. In some instances, a salt of a compound may be useful for the isolation or purification of the compound.

Where a salt is intended to be administered to a patient, the salt preferably is pharmaceutically acceptable. Pharmaceutically acceptable salts include, but are not limited to, acid addition salts, base addition salts, and alkali metal salts.

Pharmaceutically acceptable acid addition salts may be prepared from inorganic or organic acids. Examples of suitable inorganic acids include, but are not limited to, hydrochloric, hydrobromic, hydroiodic, nitric, carbonic, sulfuric, and phosphoric acid. Examples of suitable organic acids include, but are not limited to, aliphatic, cycloaliphatic, aromatic, araliphatic, heterocyclyl, carboxylic, and sulfonic classes of organic acids. Specific examples of suitable organic acids include acetate, trifluoroacetate, formate, propionate, succinate, glycolate, gluconate, digluconate, lactate, malate, tartaric acid, citrate, ascorbate, glucuronate, maleate, fumarate, pyruvate, aspartate, glutamate, benzoate, anthranilic acid, mesylate, stearate, salicylate, p-hydroxybenzoate, phenylacetate, mandelate, embonate (pamoate), methanesulfonate, ethanesulfonate, benzenesulfonate, pantothenate, toluenesulfonate, 2-hydroxyethanesulfonate, sufanilate, cyclohexylaminosulfonate, algenic acid, b-hydroxybutyric acid, galactarate, galacturonate, adipate, alginate, bisulfate, butyrate, camphorate, camphorsulfonate, cyclopentanepropionate, dodecylsulfate, glycoheptanoate, glycerophosphate, hemisulfate, heptanoate, hexanoate, nicotinate, 2-naphthalesulfonate, oxalate, palmoate, pectinate, persulfate, 3-phenylpropionate, picrate, pivalate, thiocyanate, tosylate, and undecanoate.

Pharmaceutically acceptable base addition salts include, but are not limited to, metallic salts and organic salts. Non-limiting examples of suitable metallic salts include alkali metal (group Ia) salts, alkaline earth metal (group IIa) salts, and other pharmaceutically acceptable metal salts. Such salts may be made, without limitation, from aluminum, calcium, lithium, magnesium, potassium, sodium, or zinc. Non-limiting examples of suitable organic salts can be made from tertiary amines and quaternary amine, such as tromethamine, diethylamine, N,N′-dibenzylethylene-diamine, chloroprocaine, choline, diethanolamine, ethylenediamine, meglumine (N-methylglucamine), and procaine. Basic nitrogen-containing groups can be quaternized with agents such as alkyl halides (e.g., methyl, ethyl, propyl, butyl, decyl, lauryl, myristyl, and stearyl chlorides/bromides/iodides), dialkyl sulfates (e g, dimethyl, diethyl, dibutyl, and diamyl sulfates), aralkyl halides (e.g., benzyl and phenethyl bromides), and others.

The compounds of the invention comprise asymmetrically substituted carbon atoms known as chiral centers. These compounds may exist, without limitation, as single stereoisomers (e.g., single enantiomers or single diastereomer), mixtures of stereoisomers (e.g., a mixture of enantiomers or diastereomers), or racemic mixtures. Compounds identified herein as single stereoisomers are meant to describe compounds that are present in a form that is substantially free from other stereoisomers (e.g., substantially free from other enantiomers or diastereomers). By “substantially free,” it means that at least 80% of the compound in a composition is the described stereoisomer; preferably, at least 90% of the compound in a composition is the described stereoisomer; and more preferably, at least 95%, 96%, 97%, 98% or 99% of the compound in a composition is the described stereoisomer. Where the stereochemistry of a chiral carbon is not specified in the chemical structure of a compound, the chemical structure is intended to encompass compounds containing either stereoisomer of the chiral center. Methods of stereoselective synthesis and stereoisomer resolution for the compound of formula (I) are discussed in PCT Application Publication No. WO 2012/051361.

In certain embodiments, the invention relates to a pharmaceutical composition comprising a crystalline compound or a crystalline solvate of a compound of formula (I) and one or more pharmaceutically acceptable excipients. In certain embodiments, the pharmaceutical composition is selected from tablets, capsules, and suspensions.

The term “substantially pure” as used herein, refers to a crystalline polymorph that is greater than 90% pure, meaning that contains less than 10% of any other compound, including the corresponding amorphous compound or an alternative polymorph of the crystalline compound. Preferably, the crystalline polymorph is greater than 95% pure, or even greater than 98% pure.

The term “prodrug” is intended to encompass compounds which, under physiologic conditions, are converted into the therapeutically active agents of the present invention (e.g., a compound of formula (I)). A common method for making a prodrug is to include one or more selected moieties which are hydrolyzed under physiologic conditions to reveal the desired molecule. In other embodiments, the prodrug is converted by an enzymatic activity of the host animal.

### III. Methods of Making the Crystalline Compound and Solvates

In certain embodiments, the invention relates to a method for the preparation of a first crystalline form of a compound having the structure of formula (I) or a crystalline solvate thereof, comprising a) contacting a compound of formula (I) with one or more recrystallization solvents under conditions sufficient to form a mixture comprising a compound of formula (I) and the one or more recrystallization solvents, and b) crystallizing the compound of formula (I) from the mixture comprising a compound of formula (I) and the one or more recrystallization solvents.

In certain embodiments, in a) the compound of formula (I) is amorphous. In certain such embodiments, the one or more recrystallization solvents comprise a (C₃-C₆)alkylamine. Exemplary (C₃-C₆)alkylamines include n-butylamine, propylamine, amylamine, n-hexylamine, sec-butylamine, isobutylamine. In certain preferred embodiments, the the one or more recrystallization solvents comprises n-butylamine.

In certain embodiments, crystallization of a compound of formula (I) in the presence of a (C₃-C₆)alkylamine provides access to a crystal form of a compound of formula (I) that can be used to prepare further crystal forms and crystalline solvates.

In certain embodiments, in a) the compound of formula (I) is crystalline, such as Pattern A or one of the other forms described herein. In certain embodiments, the crystalline compound of formula (I) used in a) is a crystalline solvate.

In certain embodiments, the one or more recrystallization solvents include water, ethanol, acetonitrile, diethyl ether, di-n-butylether, n-propanol, n-butanol, 2-butanol, n-pentanol, pentane, hexane, heptane, cyclohexane, acetone, ethyl acetate, isopropanol, propylamine, amylamine, n-hexylamine, sec-butylamine, or isobutylamine.

In certain embodiments, a single recrystallization solvent is used.

In certain embodiments, more than one recrystallization solvent is used. In certain such embodiments wherein more than one recrystallization solvent is used, each of the recrystallization solvents are introduced to the compound of formula (I) simultaneously, for example, as a solvent mixture. In alternative embodiments, each of the recrystallization solvents is introduced to the compound of formula (I) sequentially. For example, an amorphous or crystalline compound of formula (I) may be dissolved or suspended in a first recrystallization solvent, and then a second recrystallization solvent may be added such that a predetermined final volume ratio of first recrystallization solvent to second recrystallization solvent is achieved. In an alternative example, a second recrystallization solvent may be added until a physical change (e.g., precipitation of a crystalline form) is observed.

In certain embodiments, the mixture comprising a compound of formula (I) and the one or more recrystallization solvents is a solution. In certain such embodiments, the methods further comprise seeding the solution with a second crystalline form of the compound of formula (I). In certain embodiments, the second crystal form that is used to seed the solution is a crystalline solvate of a compound of formula (I).

In certain embodiments, the mixture comprising a compound of formula (I) and the one or more recrystallization solvents is a solution. In certain such embodiments, crystallizing the compound of formula (I) from the solution comprises bringing the solution to supersaturation to cause the compound of formula (I) to precipitate out of solution. In other embodiments, crystallizing the compound of formula (I) from the solution comprises seeding the solution with a crystalline form of the compound of formula (I), such as crystals of Pattern A, Pattern B, Pattern C, Pattern F, Pattern G, or combinations thereof.

In certain embodiments, bringing the solution comprising the compound of formula (I) to supersaturation comprises the slow addition of an anti-solvent, such as heptanes, hexanes, ethanol, diethyl ether, di-n-butyl ether, or another polar or non-polar liquid miscible with the organic solvent, allowing the solution to cool to ambient temperature or lower (with or without seeding the solution), reducing the volume of the solution, e.g., by evaporating solvent from the solution, or any combination thereof. In certain embodiments, allowing the solution to cool may be passive (e.g., allowing the solution to stand at ambient temperature) or active (e.g., cooling the solution in an ice bath or freezer). In certain embodiments, reducing the volume of the solution may be active (e.g., by heating the solvent or applying reduced pressure to the solution) or reducing the volume of the solution may be passive (e.g., by letting the solution stand under ambient conditions for slow evaporation).

In certain embodiments, the mixture comprising a compound of formula (I) and the one or more recrystallization solvents is a suspension. In certain such embodiments, crystallizing the compound of formula (I) from the suspension can include letting the suspension sit for a period of time, e.g., for a period of time sufficient to allow crystals to settle out of the suspension. In certain other embodiments, crystallizing the compound of formula (I) from the suspension can include agitating the suspension, for example with a magnetic stirbar or a glass rod, sonicating the suspension, or centrifugation of the suspension. In certain embodiments, the crystals of the compound of formula (I) form via nucleation at a nucleation site. Nucleation sites can include objects such as a stirbar, glass rod, or a suspended particle, or can alternatively include crystalline units of the compound of formula (I).

In certain embodiments, the preparation method further comprises isolating the crystals, e.g. by filtering the crystals, by decanting fluid from the crystals, or by any other suitable separation technique. In further embodiments, the preparation method further comprises washing the crystals.

In certain embodiments, the preparation method further comprises inducing crystallization. In certain embodiments, inducing precipitation or crystallization comprises secondary nucleation, wherein nucleation occurs in the presence of seed crystals or interactions with the environment (crystallizer walls, stirring impellers, sonication, etc.).

In certain embodiments, the method further comprises the step of drying the crystalline compound. In certain embodiments, drying the crystalline compound comprises heating the crystals above ambient temperature, applying to the crystals a reduced pressure, or a vacuum, or a combination thereof. In certain other embodiments, drying the crystals comprises leaving the crystals substantially untouched under ambient temperature and pressure, or “airdrying”. In certain embodiments of the invention, drying a crystalline solvate of a compound of formula (I) yields a crystalline anhydrate of formula (I).

In certain embodiments, the recrystallization solvent comprises n-butylamine, propylamine, amylamine, n-hexylamine, sec-butylamine, isobutylamine, acetonitrile, methyl ethyl ketone (MEK), methanol, ethanol, n-butanol, 2-butanol, n-pentanol, diethyl ether, di-n-butyl ether, methyl tert-butyl ether, heptane, pentane, cyclohexane, acetone, ethyl acetate, isopropanol, water, or any combination thereof. In certain preferred embodiments, the recrystallization solvent is n-butylamine, acetonitrile, methyl ethyl ketone (MEK), methanol, ethanol, diethyl ether, methyl tert-butyl ether, heptane, cyclohexane, acetone, ethyl acetate, isopropanol, water, or any combination thereof. In an example embodiment, the recrystallization solvent comprises ethanol and water. In another example embodiment, the recrystallization solvent comprises acetonitrile. In another example embodiment, the recrystallization solvent comprises methanol and diethylether. Selection of a recrystallization solvent system is well within the purview of a person of ordinary skill in the art. The skilled person would take into account the identity of the recrystallization solvent, and, in the case of a recrystallization solvent system utilizing two or more solvents, the relative ratios of the two or more solvents. Such a solvent molecule or combination of solvent molecules may become incorporated into the crystalline solvate complex of the compound of formula (I).

In certain embodiments, washing the crystals comprises washing with a liquid such as an anti-solvent, in which the crystals are not more than minimally soluble, but impurities are anticipated to be soluble. In certain embodiments, the crystals are rinsed with the solvent or solvents that are utilized in the recrystallization. In certain embodiments, the rinsing solvent or solvents are cooled prior to their use in rinsing the crystals. As used herein, “anti-solvent” means a solvent in which the crystals are insoluble, minimally soluble, or partially soluble. In practice, the addition of an anti-solvent to a solution in which the compound is dissolved reduces the solubility of the compound in solution, thereby stimulating precipitation of the compound crystals. In certain embodiments, the crystals are washed with a combination of anti-solvent and the organic solvent. In certain embodiments, the anti-solvent is water, while in other embodiments it is an alkane solvent, such as hexane or pentane, or an aromatic hydrocarbon solvent, such as benzene, toluene, or xylene. The selection of an appropriate fluid for washing the compound crystals, in which the crystals are minimally soluble and potential impurities are soluble is well within the purview of a person of ordinary skill in the art.

In certain embodiments, washing the crystals comprises washing the crystalline compound of formula (I) with a solvent or a mixture of one or more solvents, which are described above. In certain embodiments, the solvent or mixture of solvents is cooled prior to washing.

In certain embodiments, the crystallization methods described herein enable the purification and isolation of a compound of formula (I).

In further embodiments, the methods of the invention relate to the preparation of an anhydrous crystalline form of a compound having the structure of formula (I), comprising drying a solvated crystalline form of a compound having the structure of formula (I).

In certain embodiments, drying the solvated crystalline compound occurs under reduced pressure. In certain embodiments, drying the solvated crystalline compound comprises heating the solvated crystalline compound. In further embodiments, drying the solvated crystalline compound occurs by heating under reduced pressure.

In certain embodiments, the solvated crystalline compound is a methanol-diethyl ether solvate. In alternative embodiments, the solvated crystalline compound is an acetonitrile solvate. In preferred embodiments, the resultant anhydrate is a compound as described in Section II of this specification.

### IV. Uses of Anti-Viral Compounds

In certain embodiments, the invention relates to methods of treating HCV infection comprising administering a compound of formula (I). The compound of formula (I) may be in crystalline form as described herein.

The term “treating” includes prophylactic and/or therapeutic treatments. The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic (i.e., it protects the host against developing the unwanted condition), whereas if it is administered after manifestation of the unwanted condition, the treatment is therapeutic (i.e., it is intended to diminish, ameliorate, or stabilize the existing unwanted condition or side effects thereof).

### V. Pharmaceutical Compositions

In certain embodiments, the present invention relates to pharmaceutical compositions comprising a compound of formula (I) and one or more pharmaceutically acceptable excipients. In certain embodiments, the compound of formula (I) is crystalline, or a crystalline solvate thereof. In certain embodiments, the pharmaceutical composition comprises pharmaceutically acceptable salts, solvates, or produgs of the compound of formula (I). Without limitation, pharmaceutically acceptable salts can be zwitterions or derived from pharmaceutically acceptable inorganic or organic acids or bases. Preferably, a pharmaceutically acceptable salt retains the biological effectiveness of the free acid or base of the compound without undue toxicity, irritation, or allergic response, has a reasonable benefit/risk ratio, is effective for the intended use, and is not biologically or otherwise undesirable.

A pharmaceutical composition containing multiple active ingredients can be either a co-formulated product, a co-packaged product, or a combination thereof.

A pharmaceutical composition of the present invention typically includes a pharmaceutically acceptable carrier or excipient. Non-limiting examples of suitable pharmaceutically acceptable carriers/excipients include sugars (e.g., lactose, glucose or sucrose), starches (e.g., corn starch or potato starch), cellulose or its derivatives (e.g., sodium carboxymethyl cellulose, ethyl cellulose or cellulose acetate), oils (e.g., peanut oil, cottonseed oil, safflower oil, sesame oil, olive oil, corn oil or soybean oil), glycols (e.g., propylene glycol), buffering agents (e.g., magnesium hydroxide or aluminum hydroxide), agar, alginic acid, powdered tragacanth, malt, gelatin, talc, cocoa butter, pyrogen-free water, isotonic saline, Ringer's solution, ethanol, or phosphate buffer solutions. Lubricants, coloring agents, releasing agents, coating agents, sweetening, flavoring or perfuming agents, preservatives, or antioxidants can also be included in a pharmaceutical composition of the present invention.

The pharmaceutical compositions of the present invention can be formulated based on their routes of administration using methods well known in the art. For example, a sterile injectable preparation can be prepared as a sterile injectable aqueous or oleagenous suspension using suitable dispersing or wetting agents and suspending agents. Suppositories for rectal administration can be prepared by mixing drugs with a suitable nonirritating excipient such as cocoa butter or polyethylene glycols which are solid at ordinary temperatures but liquid at the rectal temperature and will therefore melt in the rectum and release the drugs. Solid dosage forms for oral administration can be capsules, tablets, pills, powders or granules. In such solid dosage forms, the active compounds can be admixed with at least one inert diluent such as sucrose lactose or starch. Solid dosage forms may also comprise other substances in addition to inert diluents, such as lubricating agents. In the case of capsules, tablets and pills, the dosage forms may also comprise buffering agents. Tablets and pills can additionally be prepared with enteric coatings. Liquid dosage forms for oral administration can include pharmaceutically acceptable emulsions, solutions, suspensions, syrups or elixirs containing inert diluents commonly used in the art. Liquid dosage forms may also comprise wetting, emulsifying, suspending, sweetening, flavoring, or perfuming agents. The pharmaceutical compositions of the present invention can also be administered in the form of liposomes, as described in U.S. Pat. No. 6,703,403. Formulation of drugs that are applicable to the present invention is generally discussed in, for example, Hoover, John E., REMINGTON'S PHARMACEUTICAL SCIENCES (Mack Publishing Co., Easton, Pa.: 1975), and Lachman, L., eds., PHARMACEUTICAL DOSAGE FORMS (Marcel Decker, New York, N.Y., 1980).

Any compound described herein, or a pharmaceutically acceptable salt thereof, can be used to prepared pharmaceutical compositions of the present invention.

In a preferred embodiment, a compound of the invention (e.g., a compound of Formula (I), or preferably a compound described hereinabove, or a salt, solvate or prodrug thereof) is formulated in a solid dispersion, where the compound of the invention can be molecularly dispersed in an amorphous matrix which comprises a pharmaceutically acceptable, hydrophilic polymer. The matrix may also contain a pharmaceutically acceptable surfactant. Suitable solid dispersion technology for formulating a compound of the invention includes, but is not limited to, melt-extrusion, spray-drying, co-precipitation, freeze drying, or other solvent evaporation techniques, with melt-extrusion and spray-drying being preferred. In one example, a compound of the invention is formulated in a solid dispersion comprising copovidone and vitamin E TPGS. In another example, a compound of the invention is formulated in a solid dispersion comprising copovidone and Span 20.

A solid dispersion described herein may contain at least 30% by weight of a pharmaceutically acceptable hydrophilic polymer or a combination of such hydrophilic polymers. Preferably, the solid dispersion contains at least 40% by weight of a pharmaceutically acceptable hydrophilic polymer or a combination of such hydrophilic polymers. More preferably, the solid dispersion contains at least 50% (including, e.g., at least 60%, 70%, 80% or 90%) by weight of a pharmaceutically acceptable hydrophilic polymer or a combination of such polymers. A solid dispersion described herein may also contain at least 1% by weight of a pharmaceutically acceptable surfactant or a combination of such surfactants. Preferably, the solid dispersion contains at least 2% by weight of a pharmaceutically acceptable surfactant or a combination of such surfactants. More preferably, the solid dispersion contains from 4% to 20% by weight of the surfactant(s), such as from 5% to 10% by weight of the surfactant(s). In addition, a solid dispersion described herein may contain at least 1% by weight of a compound of the invention, preferably at least 5%, including, e.g., at least 10%. In one example, the solid dispersion comprises 5% of a compound of the invention (e.g., a compound of Formula (I), or preferably a compound described hereinabove, or a salt, solvate or prodrug thereof), which is molecularly dispersed in a an amorphous matrix comprising 7% Vitamin E-TPGS and 88% copovidone; the solid dispersion can also be mixed with other excipients such as mannitol/aerosil (99:1), and the weight ratio of the solid dispersion over the other excipients can range from 5:1 to 1:5 with 1:1 being preferred. In another example, the solid dispersion comprises 5% of a compound of the invention (e.g., a compound of Formula (I), or preferably a compound described hereinabove, or a salt, solvate or prodrug thereof), which is molecularly dispersed in a an amorphous matrix comprising 5% Span 20 and 90% copovidone; the solid dispersion can also be mixed with other excipients such as mannitol/aerosil (99:1), the solid dispersion can also be mixed with other excipients such as mannitol/aerosil (99:1), and the weight ratio of the solid dispersion over the other excipients can range from 5:1 to 1:5 with 1:1 being preferred.

Various additives can also be included in or mixed with the solid dispersion. For instance, at least one additive selected from flow regulators, binders, lubricants, fillers, disintegrants, plasticizers, colorants, or stabilizers may be used in compressing the solid dispersion to tablets. These additives can be mixed with ground or milled solid dispersion before compacting. Disintegrants promote a rapid disintegration of the compact in the stomach and keeps the liberated granules separate from one another. Non-limiting examples of suitable disintegrants are cross-linked polymers such as cross-linked polyvinyl pyrrolidone, cross-linked sodium carboxymethylcellulose or sodium croscarmellose. Non-limiting examples of suitable fillers (also referred to as bulking agents) are lactose monohydrate, calcium hydrogenphosphate, microcrystalline cellulose (e.g., Avicell), silicates, in particular silicium dioxide, magnesium oxide, talc, potato or corn starch, isomalt, or polyvinyl alcohol. Non-limiting examples of suitable flow regulators include highly dispersed silica (e.g., colloidal silica such as Aerosil), and animal or vegetable fats or waxes. Non-limiting examples of suitable lubricants include polyethylene glycol (e.g., having a molecular weight of from 1000 to 6000), magnesium and calcium stearates, sodium stearyl fumarate, and the like. Non-limiting examples of stabilizers include antioxidants, light stabilizers, radical scavengers, or stabilizers against microbial attack.

The present invention further features methods of using the compounds of the present invention (or salts, solvates or prodrugs thereof) to inhibit HCV replication. The methods comprise contacting cells infected with HCV virus with an effective amount of a compound of the present invention (or a salt, solvate or prodrug thereof), thereby inhibiting c replication of HCV virus in the cells. As used herein, “inhibiting” means significantly reducing, or abolishing, the activity being inhibited (e.g., viral replication). In many cases, representative compounds of the present invention can reduce the replication of HCV virus (e.g., in an HCV replicon assay as described above) by at least 10%, 20%, 30%, 40%, 50%, 60%, 70%, 80%, 90%, 95% or more.

The compounds of the present invention may inhibit one or more HCV subtypes. Examples of HCV subtypes that are amenable to the present invention include, but are not be limited to, HCV genotypes 1, 2, 3, 4, 5 and 6, including HCV genotypes 1a, 1b, 2a, 2b, 2c, 3a or 4a. In one embodiment, a compound or compounds of the present invention (or salts, solvates or prodrugs thereof) are used to inhibit the replication of HCV genotype 1b. In another embodiment, a compound or compounds of the present invention (or salts, solvates or prodrugs thereof) are used to inhibit the replication of HCV genotype 1b. In still another embodiment, a compound or compounds of the present invention (or salts, solvates or prodrugs thereof) are used to inhibit the replication of both HCV genotypes 1a and 1b.

The present invention also features methods of using the compounds of the present invention (or salts, solvates or prodrugs thereof) to treat HCV infection. The methods typically comprise administering a therapeutic effective amount of a compound of the present invention (or a salt, solvate or prodrug thereof), or a pharmaceutical composition comprising the same, to an HCV patient, thereby reducing the HCV viral level in the blood or liver of the patient. As used herein, the term “treating” refers to reversing, alleviating, inhibiting the progress of, or preventing the disorder or condition, or one or more symptoms of such disorder or condition to which such term applies. The term “treatment” refers to the act of treating. In one embodiment, the methods comprise administering a therapeutic effective amount of two or more compounds of the present invention (or salts, solvates or prodrugs thereof), or a pharmaceutical composition comprising the same, to an HCV patient, thereby reducing the HCV viral level in the blood or liver of the patient.

A compound of the present invention (or a salt, solvate or prodrug thereof) can be administered as the sole active pharmaceutical agent, or in combination with another desired drug, such as other anti-HCV agents, anti-HIV agents, anti-HBV agents, anti-hepatitis A agents, anti-hepatitis D agents, anti-hepatitis E agents, anti-hepatitis G agents, or other antiviral drugs. Any compound described herein, or a pharmaceutically acceptable salt thereof, can be employed in the methods of the present invention. In one embodiment, the present invention features methods of treating HCV infection, wherein said methods comprise administering a compound of the invention (e.g., a compound of Formula (I), or preferably a compound described hereinabove, or a salt, solvate or prodrug thereof), interferon and ribavirin to an HCV patient. The interferon preferably is α-interferon, and more preferably, pegylated interferon-α such as PEGASYS (peginterferon alfa-2a).

In another embodiment, the present invention features methods of treating HCV infection, wherein said methods comprise administering a compound of the invention (e.g., a compound of Formula (I), or a salt, solvate or prodrug thereof), and one or more HCV inhibitors/modulators described above, with or without interferon.

A compound of the present invention (or a salt, solvent or prodrug thereof) can be administered to a patient in a single dose or divided doses. A typical daily dosage can range, without limitation, from 0.1 to 200 mg/kg body weight, such as from 0.25 to 100 mg/kg body weight. Single dose compositions can contain these amounts or submultiples thereof to make up the daily dose. Preferably, each dosage contains a sufficient amount of a compound of the present invention that is effective in reducing the HCV viral load in the blood or liver of the patient. The amount of the active ingredient, or the active ingredients that are combined, to produce a single dosage form may vary depending upon the host treated and the particular mode of administration. It will be understood that the specific dose level for any particular patient will depend upon a variety of factors including the activity of the specific compound employed, the age, body weight, general health, sex, diet, time of administration, route of administration, rate of excretion, drug combination, and the severity of the particular disease undergoing therapy.

The present invention further features methods of using the pharmaceutical compositions of the present invention to treat HCV infection. The methods typically comprise administering a pharmaceutical composition of the present invention to an HCV patient, thereby reducing the HCV viral level in the blood or liver of the patient. Any pharmaceutical composition described herein can be used in the methods of the present invention.

In addition, the present invention features use of the compounds or salts of the present invention for the manufacture of medicaments for the treatment of HCV infection. Any compound described herein, or a pharmaceutically acceptable salt thereof, can be used to make medicaments of the present invention.

The invention now being generally described, it will be more readily understood by reference to the following examples which are included merely for purposes of illustration of certain aspects and embodiments of the present invention, and are not intended to limit the invention.

### EXAMPLES

### Example 1

### Synthetic Protocols for the Compound of Formula (I)

Synthetic protocols and characterization data for making the amorphous compounds of formula (I) may be found in PCT Application Publication No. WO 2012/051361.

### Example 2

### Preparation of Crystal Forms of the Compounds of Formula (I)

n-Butylamine-H₂O Solvate (Pattern A)

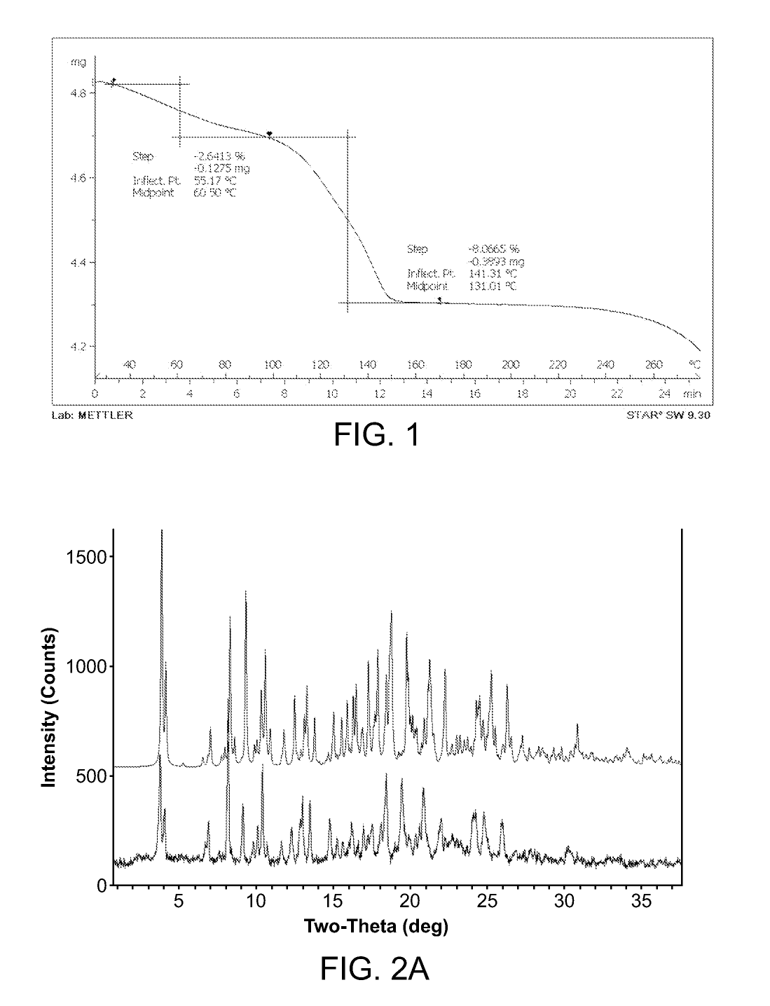

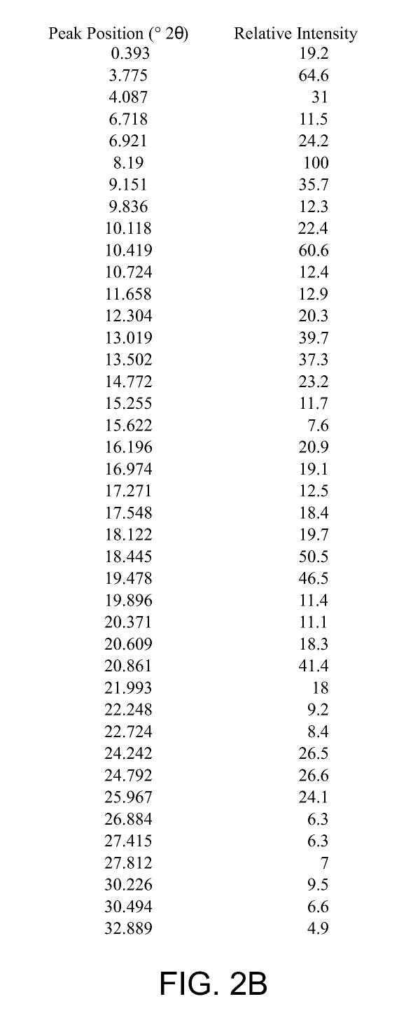

Amorphous 1 (the compound of formula (I)) was suspended in n-butylamine at ambient temperature. Solids were isolated after crystallization and left at ambient conditions for a short period of time prior to characterization. The crystal structure was resolved by Single-Crystal XRD. The asymmetric unit contained 4 molecules of n-butylamine, 2 molecules of water and 2 molecules of 1. The experimental and calculated (from Single-Crystal XRD data) Powder X-ray diffraction patterns are shown in FIG. 2. Peak listing of the experimental PXRD pattern with relative intensities is given in FIG. 2B. Thermogravimetric analysis (TGA) is shown in FIG. 1.

Isostructural crystal forms (Pattern A) were obtained from other solvent systems, including propylamine/H₂O, amylamine, n-hexylamine, sec-butylamine/H₂O, isobutylamine/H₂O, n-butanol/Heptane, 2-butanol/Heptane, n-pentanol/Heptane, EtOH/Pentane and n-propanol/Pentane.

### Methyl Ethyl Ketone-Heptane Solvate (Pattern B)

Amorphous 1 was dissolved in methyl ethyl ketone (MEK) at ambient temperature and heptane was added. A seed mixture was prepared from different crystalline solids of 1 including Pattern A (n-butylamine-H₂O solvate) and other pattern A forms isolated from alkylamines. Solids were isolated after crystallization and left at ambient conditions for a short period of time prior to characterization.

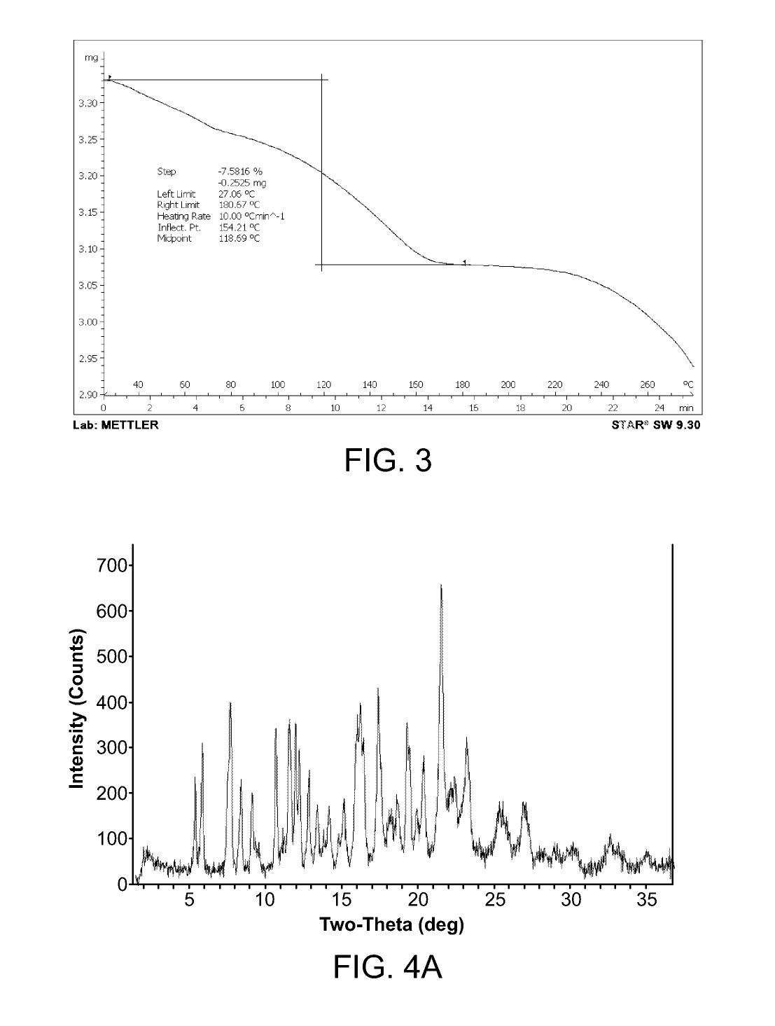

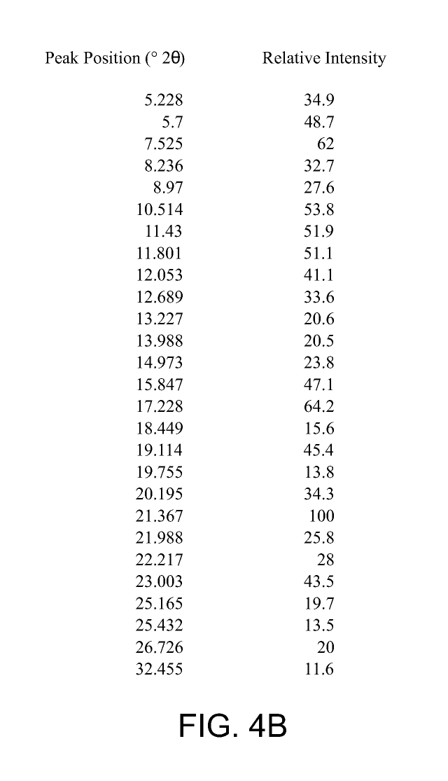

Powder X-ray diffraction pattern and peak listing with relative intensities are shown in FIG. 4A-B. Thermogravimetric analysis (TGA) is shown in FIG. 3.

### MeOH/Diethyl Ether Solvate (Pattern B)

Amorphous 1 was dissolved in methanol at ambient temperature and diethyl ether was added. Pattern B seeds were added to the solution. Solids were isolated after crystallization and left at ambient conditions for a short period of time prior to characterization.

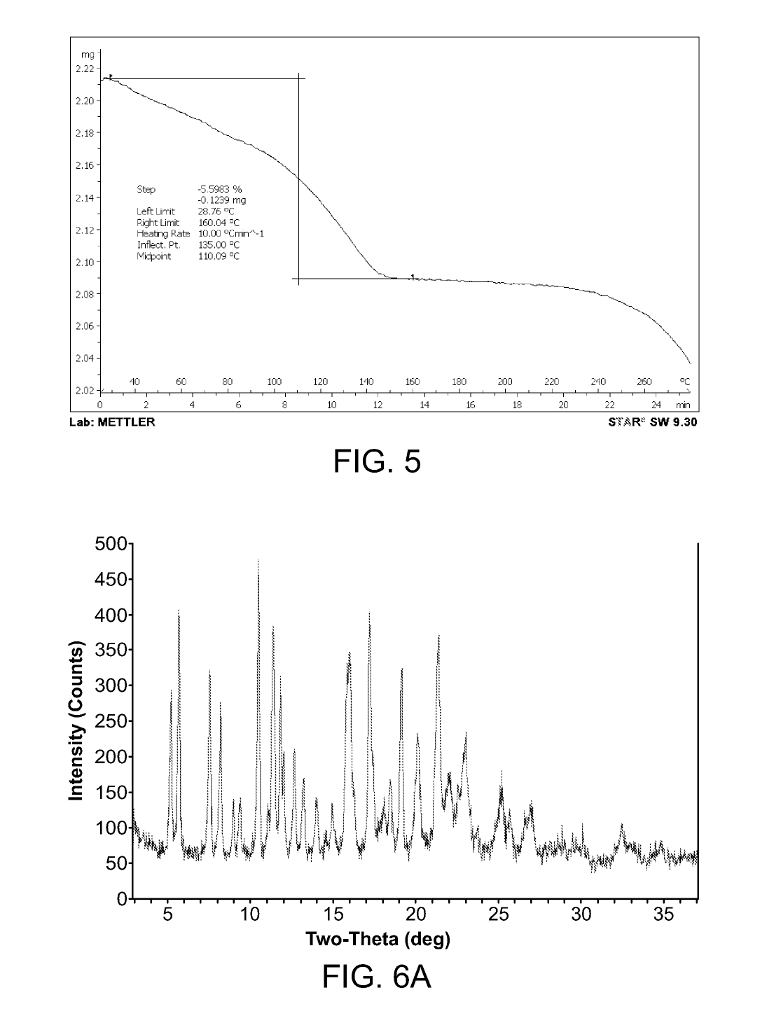

Powder X-ray diffraction pattern and peak listing with relative intensities are shown in FIGS. 6A-B. Thermogravimetric analysis (TGA) is shown in FIG. 5.

Isostructural crystal forms (Pattern B) were obtained from over 15 other solvent systems. All crystal forms characterized as Pattern B have been solvates.

### Anhydrate (Pattern C)

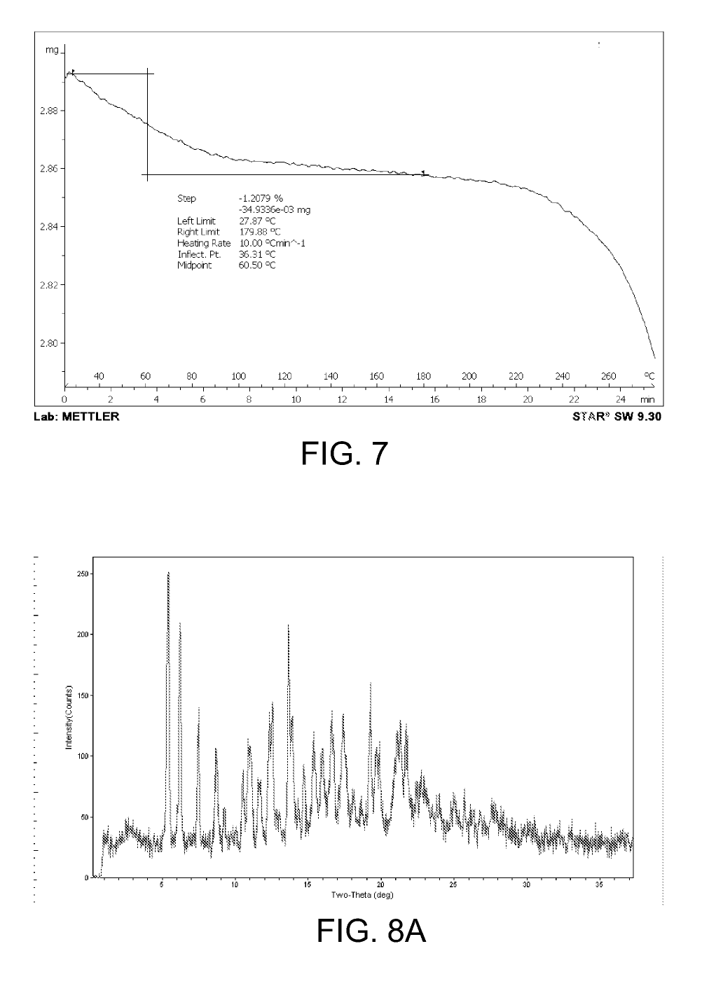

The Pattern B methanol-diethyl ether solvate of 1 was dried under vacuum at 50° C. for two weeks. Solids were equilibrated a short time prior to characterization. Powder X-ray diffraction pattern and peak listing with relative intensities are shown in FIGS. 8A-B. Thermogravimetric analysis (TGA) is shown in FIG. 7.

### MTBE Solvate (Pattern C)

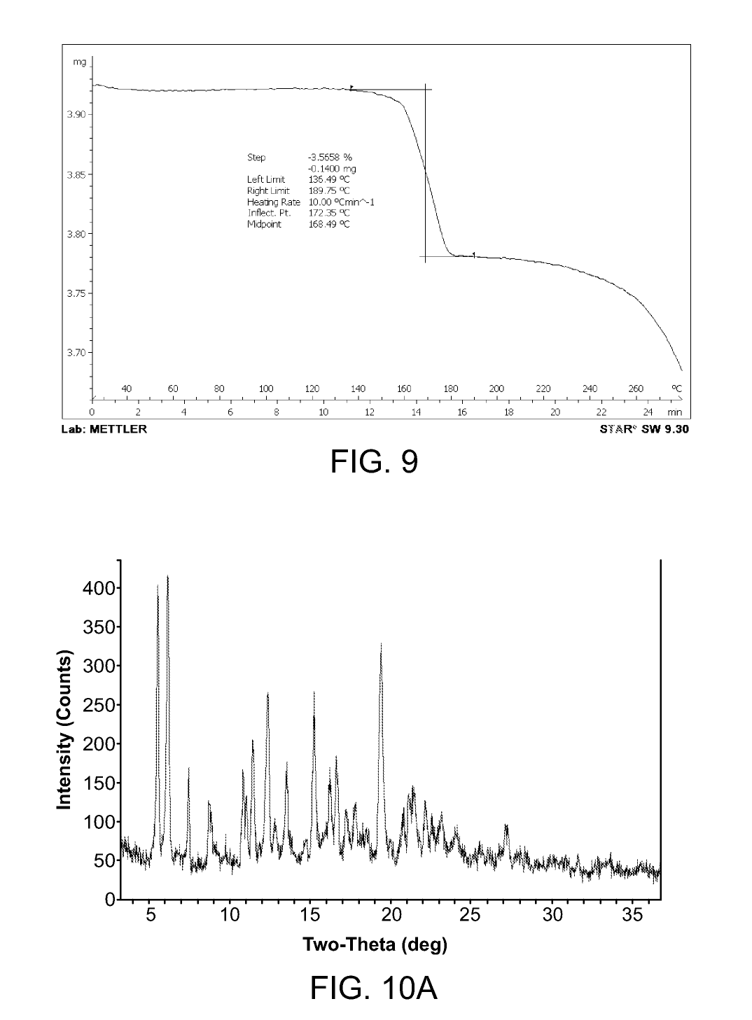

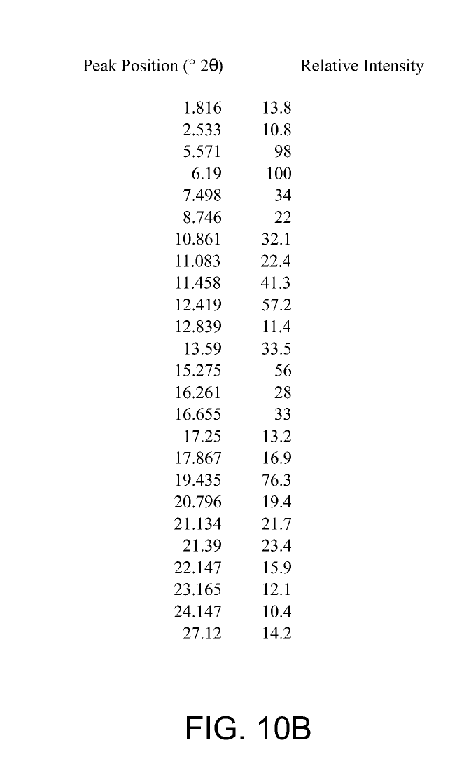

The Pattern B MTBE solvate of 1 was dried under vacuum at 70° C. for two weeks. Solids were equilibrated a short time prior to characterization. Powder X-ray diffraction pattern and peak listing with relative intensities are shown in FIGS. 10A-B. Thermogravimetric analysis (TGA) is shown in FIG. 9.

### Ethanol-H₂O Solvate (Pattern D)

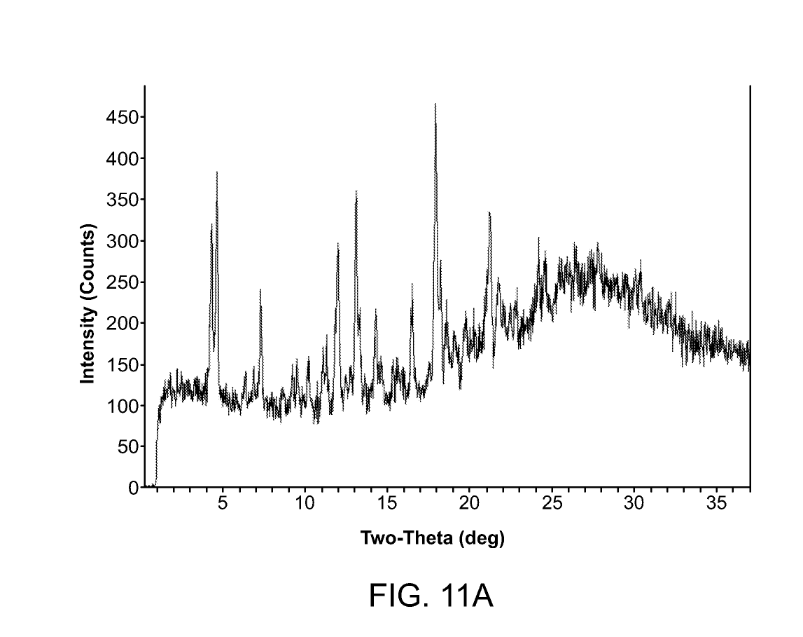

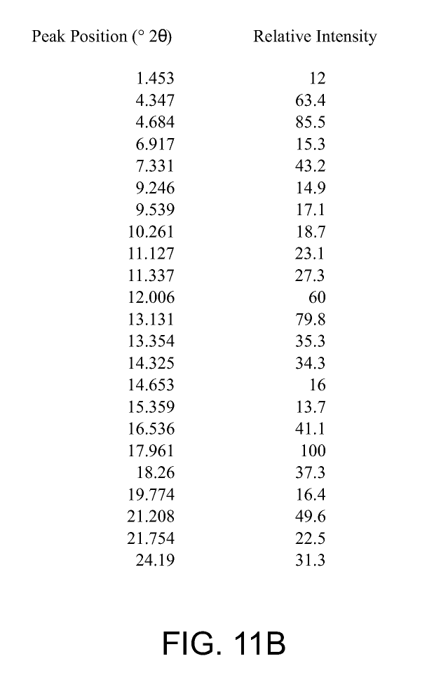

1 Pattern B MTBE solvate and Pattern C MTBE solvate were combined and suspended in 20 wt % ethanol in H₂O at ambient temperature for approximately three weeks. Solids were analyzed by PXRD while still wet. Powder X-ray diffraction pattern and peak listing with relative intensities are shown in FIGS. 11A-B.

### Hydrate (Pattern E)

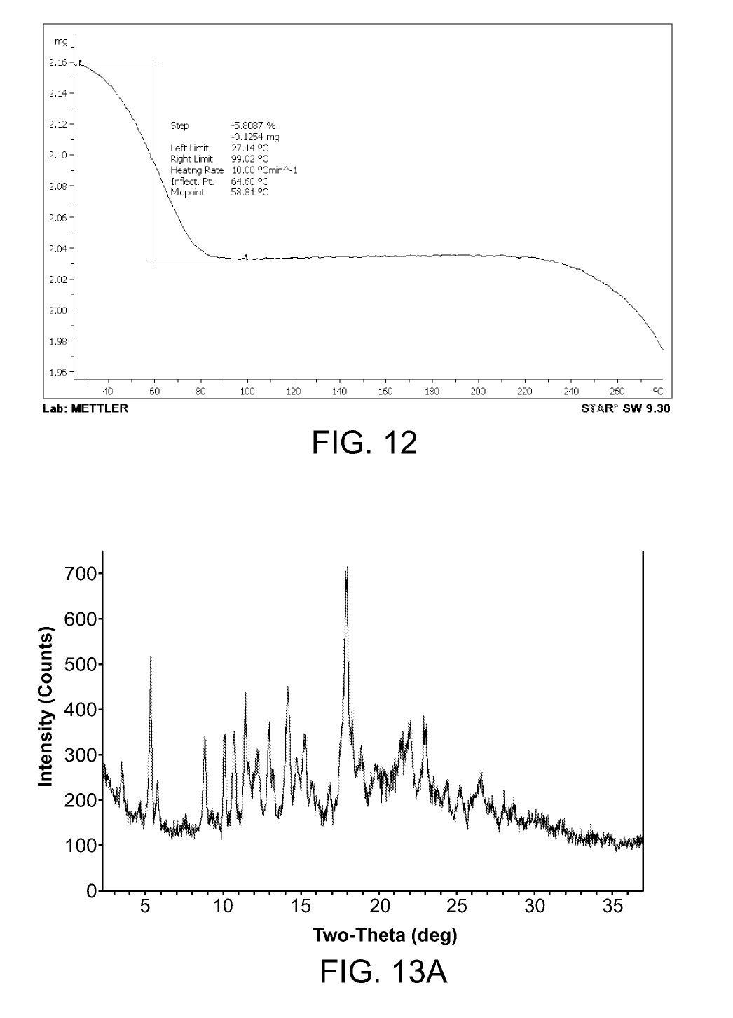

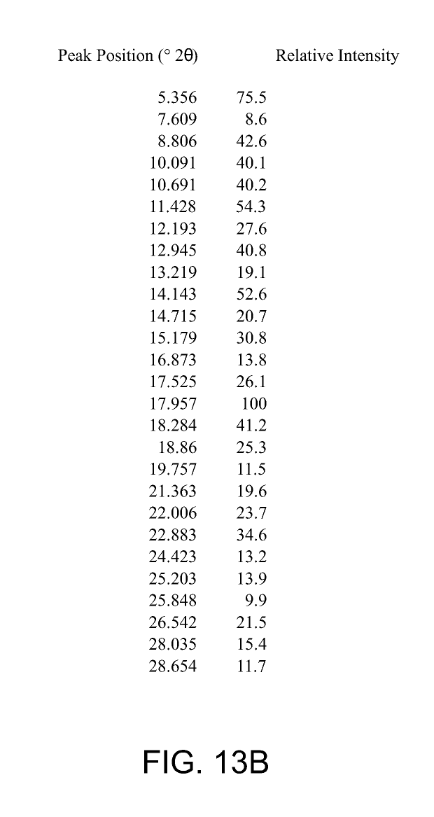

The Pattern D ethanol-H₂O solvate of 1 was airdried for approximately 2 h. Powder X-ray diffraction pattern and peak listing with relative intensities are shown in FIGS. 13A-B. Thermogravimetric analysis (TGA) is shown in FIG. 12.

### Acetonitrile Solvate (Pattern F)

The Pattern B MTBE solvate of 1 was suspended in acetonitrile at ambient temperature over four days. Solids were analyzed by PXRD while still wet. Powder X-ray diffraction pattern and peak listing with relative intensities are shown in FIGS. 14A-B.

### Acetonitrile-Di-n-Butyl Ether Solvate (Pattern F)

1 was dissolved in acetonitrile at 40° C. Di-n-butyl ether was charged to prepare a 60% di-n-butyl ether/acetonitrile composition, and the solution was seeded with Pattern G anhydrate. The mixture was charged with di-n-butyl ether to a composition of 83% di-n-butyl ether/acetonitrile and cooled to 25° C. Solids were analyzed by PXRD while still wet.

### Anhydrate (Pattern G)

The Pattern F acetonitrile solvate of 1 or the Pattern F acetonitrile-di-n-butyl ether solvate of 1 was airdried at ambient temperature for a few minutes. Powder X-ray diffraction pattern and peak listing with relative intensities are shown in FIGS. 16A-B. Thermogravimetric analysis (TGA) is shown in FIG. 15.

### Di-n-Butyl Ether Solvate (Pattern H)

Pattern G and Pattern C solids in an ˜1:1 ratio were suspended in di-n-butyl ether at 25° C. for ˜3 months. Solids were analyzed by PXRD after a short equilibration time at ambient temperature.

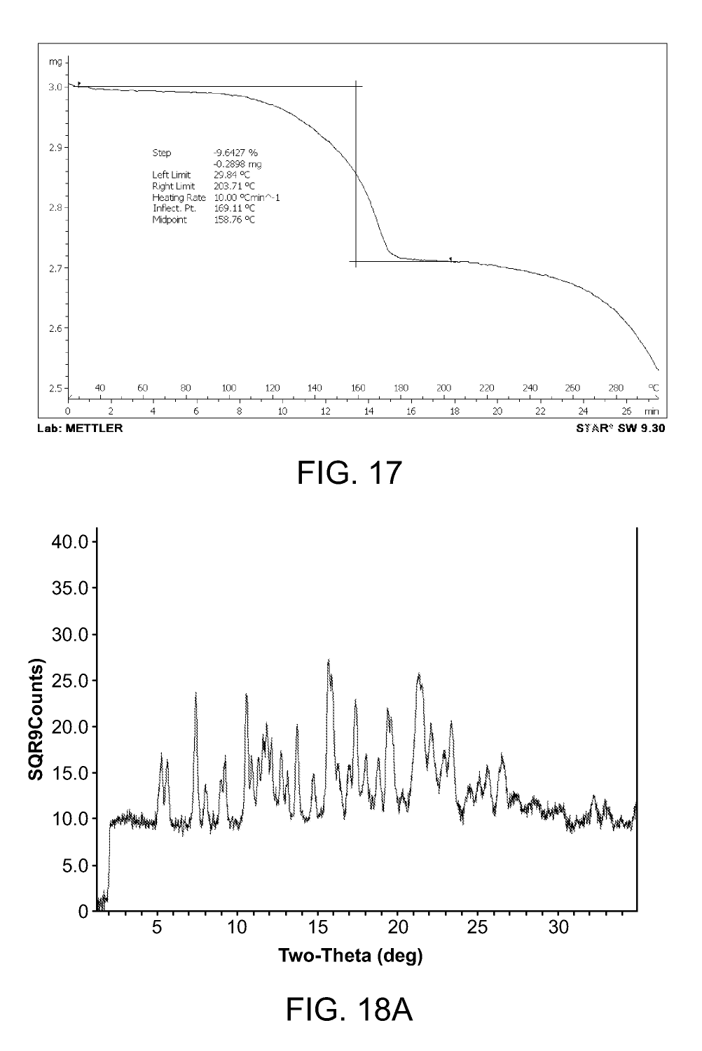

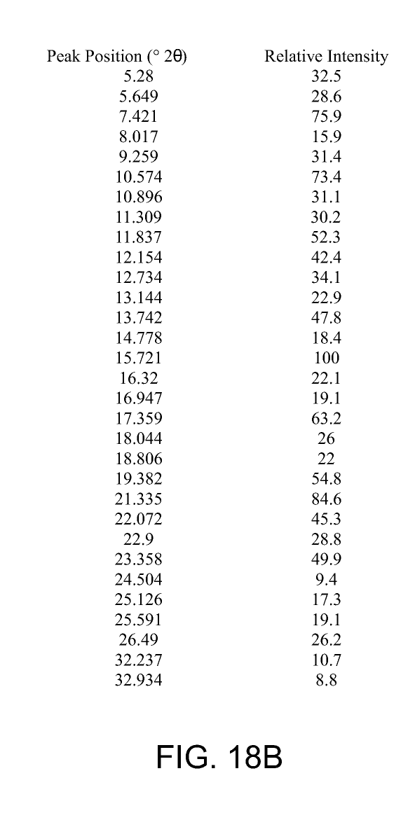

Powder X-ray diffraction pattern and peak listing with relative intensities are shown in FIGS. 18A-B. Thermogravimetric analysis (TGA) is shown in FIG. 17.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A crystalline form of a compound having the structure of formula (I),

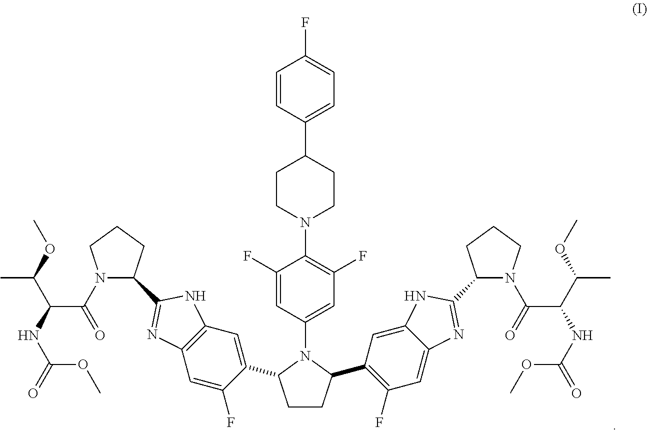 [SMILES: COC(=O)N[C@H](C(=O)N1CCC[C@H]1c1nc2cc(F)c([C@H]3CC[C@H](c4cc5[nH]c([C@@H]6CCCN6C(=O)[C@@H](NC(=O)OC)[C@@H](C)OC)nc5cc4F)N3c3cc(F)c(N4CCC(c5ccc(F)cc5)CC4)c(F)c3)cc2[nH]1)[C@@H](C)OC]

2. The crystalline form of claim 1, wherein the crystalline form is an anhydrate.

3. The crystalline form of claim 1, having 2θ values 5.31; 12.60; 13.75; 17.62; and 21.30.

4. (canceled)

5. (canceled)

6. (canceled)

7. The crystalline form of claim 3, having an XRD pattern substantially as shown in FIG. 16A.

8. The crystalline form of claim 1, having 2θ values 5.43; 6.24; 7.53; 13.67; and 19.30.

9. (canceled)

10. (canceled)

11. (canceled)

12. The crystalline form of claim 8, having an XRD pattern substantially as shown in FIG. 8A.

13. The crystalline form of claim 1, wherein the crystalline form is a solvate.

14. The crystalline form of claim 13, wherein the solvate comprises one or more solvent compounds selected from n-butylamine, propylamine, amylamine, n-hexylamine, sec-butylamine, isobutylamine, acetonitrile, methyl ethyl ketone (MEK), methanol, ethanol, n-butanol, 2-butanol, n-pentanol, diethyl ether, di-n-butyl ether, methyl tert-butyl ether, heptane, pentane, cyclohexane, acetone, ethyl acetate, isopropanol, and water.

15. (canceled)

16. The crystalline form of claim 14, wherein the solvate comprises acetonitrile and optionally di-n-butyl ether.

17. The crystalline form of claim 14, having 2θ values 5.08; 12.05; 21.73; and 25.53.

18. (canceled)

19. (canceled)

20. (canceled)

21. The crystalline form of claim 17, having an XRD pattern substantially as shown in FIG. 14A.

22. The crystalline form of claim 14, wherein the solvate comprises methanol and diethylether.

23. The crystalline form of claim 22, having 2θ values 5.69; 10.49; 11.38; 17.23; and 21.41.

24. (canceled)

25. (canceled)

26. (canceled)

27. The crystalline form of claim 23, having an XRD pattern substantially as shown in FIG. 6A.

28. A pharmaceutical composition comprising the crystalline form of claim 1; and one or more pharmaceutically acceptable excipients.

29. A method for preparing a first crystalline form of a compound having the structure of formula (I) 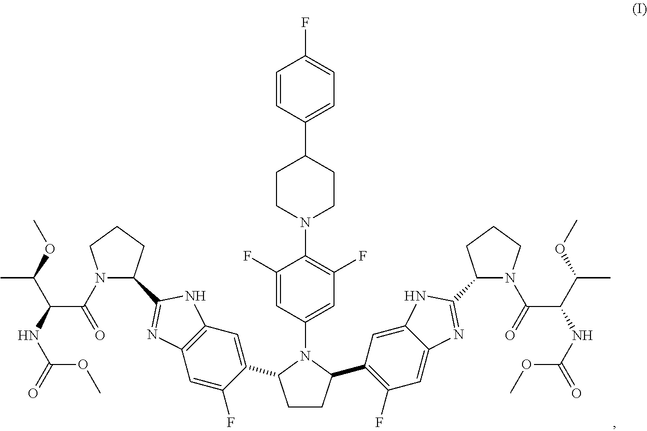 [SMILES: COC(=O)N[C@H](C(=O)N1CCC[C@H]1c1nc2cc(F)c([C@H]3CC[C@H](c4cc5[nH]c([C@@H]6CCCN6C(=O)[C@@H](NC(=O)OC)[C@@H](C)OC)nc5cc4F)N3c3cc(F)c(N4CCC(c5ccc(F)cc5)CC4)c(F)c3)cc2[nH]1)[C@@H](C)OC]
comprising:
a) contacting a compound of formula (I) with one or more recrystallization solvents under conditions sufficient to form a mixture comprising a compound of formula (I) and one or more recrystallization solvents; and
b) crystallizing the compound of formula (I) from the mixture.

30-50. (canceled)

51. A method for preparing an anhydrous crystalline form of a compound having the structure of formula (I) 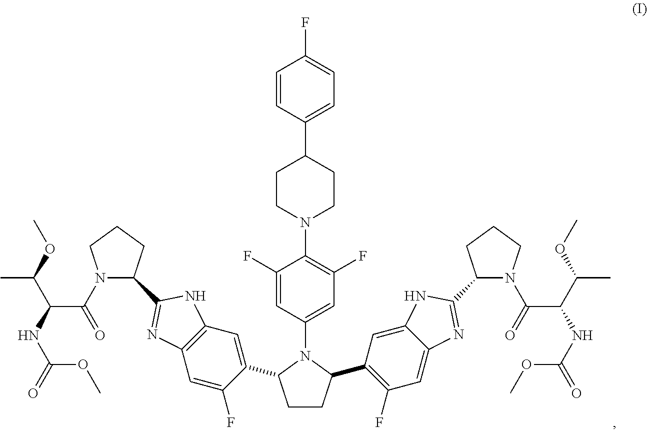 [SMILES: COC(=O)N[C@H](C(=O)N1CCC[C@H]1c1nc2cc(F)c([C@H]3CC[C@H](c4cc5[nH]c([C@@H]6CCCN6C(=O)[C@@H](NC(=O)OC)[C@@H](C)OC)nc5cc4F)N3c3cc(F)c(N4CCC(c5ccc(F)cc5)CC4)c(F)c3)cc2[nH]1)[C@@H](C)OC]
comprising drying a solvated crystalline form of a compound having the structure of formula (I).

52-55. (canceled)
